(19) United States
(12) Patent Application Publication
(10) Pub. No.: US 2026/0335551 A1
(43) Pub. Date: Oct. 29, 2026

(54) COMPACT FLUID DELIVERY SYSTEM

(71) Applicant: INNOCAT LTD., Givat Ela (IL)

(72) Inventors: Ishai BEN DAVID, Givat Ela (IL); Yair EDELSTEIN, Givat Ela (IL)

(21) Appl. No.: 19/113,360

(22) PCT Filed: Sep. 27, 2023
(86) PCT No.: PCT/IL2023/051040

§ 371 Date: Mar. 19, 2025

Related U.S. Application Data
(60) Provisional application No. 63/411,139, filed on Sep. 29, 2022.
(60) Provisional application No. 63/411,144, filed on Sep. 29, 2022.
(60) Provisional application No. 63/411,147, filed on Sep. 29, 2022.
(60) Provisional application No. 63/411,151, filed on Sep. 29, 2022.
(60) Provisional application No. 63/411,153, filed on Sep. 29, 2022.
(60) Provisional application No. 63/411,155, filed on Sep. 29, 2022.
(60) Provisional application No. 63/411,158, filed on Sep. 29, 2022.
(60) Provisional application No. 63/411,160, filed on Sep. 29, 2022.
(60) Provisional application No. 63/411,162, filed on Sep. 29, 2022.

(51) Int. Cl. A61M 5/20; A61M 5/24

(52) U.S. Cl. CPC A61M 5/20; A61M 5/2422; A61M 2205/33; A61M 2205/70

(57) ABSTRACT

A system for fluid delivery includes a propulsion unit, configured to be positioned against a plunger in a barrel of a cartridge, to eject a fluid from the cartridge. The propulsion unit has a motor shaft aligned coaxially with the cartridge. A motor shaft gear is affixed to the motor shaft and multiple wheel extensions are arranged around the motor shaft, each wheel extension having a secondary gear, one or more wheels, and a wheel gear affixed to the one or more wheels, such that rotation of the motor shaft in a first direction unfolds the wheel extensions until the wheels of the multiple wheel extensions are in contact with an inner wall of the cartridge, after which rotation of the motor shaft rotates the wheels along the inner wall to propel the propulsion unit and drive the plunger forward.

### FIELD OF THE INVENTION

The present invention relates generally to the field of devices for fluid delivery, in particular by a wearable system for delivery of a medication.

### BACKGROUND

Wearable fluid delivery devices are becoming increasingly popular, due to the convenience they provide to patients who require drug and/or supplement injections on a regular a basis. Such devices may be affixed to the body of a patient to release a fluid (or like injectable material) over a period of time. Delivery may be intravenous (IV), intramuscular (IM), or subcutaneous (SQ). Similarly configured fluid delivery devices also have additional applications, such as for wet lab research, industrial applications, and any other application requiring accurate, small scale fluid delivery.

A fluid delivery device is described in International Patent Publication WO2021/099992 A2 to Ben David, the inventor of the present invention. The fluid delivery device includes a mechanism that has a central longitudinal axis and is arranged to move forward in an axial direction. The mechanism includes a housing and one or more swing members pivotally coupled to the housing that are arranged to swing relative to the housing between an initial position and a terminal position.

A more compact fluid delivery devices could improve convenience and usability for a range of applications.

### SUMMARY

Embodiments of the present invention provide a system and methods for fluid delivery, and particularly for delivery of a medication such as insulin or a nutrient supplement to a patient, such as by injection from a wearable device. The system may include a propulsion unit for driving a plunger in a barrel of a cartridge to eject a fluid from the cartridge. The propulsion unit may include a bi-directional motor having a motor shaft rotatable in first and second directions, and the propulsion unit may be configured to be positioned in the cartridge barrel with the motor shaft aligned coaxially with the cartridge. The propulsion unit may also include a motor shaft gear affixed to the motor shaft and multiple wheel extensions arranged around the motor shaft, such that rotation of the motor shaft in a first direction unfolds the wheel extensions until the wheels of the multiple wheel extensions are in contact with an inner wall of the cartridge, and such that, when the wheels are in contact with the inner wall of the cartridge, the rotation of the motor shaft in the first direction rotates the wheels to propel the propulsion unit, driving the plunger forward. Each wheel extension may comprise a secondary gear, one or more wheels and a wheel gear mounted to an axle of the one or more wheels. The secondary gear of each wheel extension may mesh with the motor shaft gear and with the wheel gear.

### BRIEF DESCRIPTION OF DRAWINGS

For a better understanding of various embodiments of the invention and to show how the same may be carried into effect, reference will now be made, by way of example, to the accompanying drawings. Structural details of the invention are shown to provide a fundamental understanding of the invention, the description, taken with the drawings, making apparent to those skilled in the art how the several forms of the invention may be embodied in practice.

In the accompanying drawings:

FIGS. 1, 2A and 2B are schematic illustrations of a system for fluid delivery, according to some embodiments of the present invention;

FIG. 3 is a schematic illustration of a drive section of the system, according to some embodiments of the present invention;

FIG. 4 is a schematic illustration of a fluid cartridge of the system, according to some embodiments of the present invention;

FIG. 5 is a schematic illustration of a delivery section of the system, according to some embodiments of the present invention;

FIGS. 6A, 6B and 7 show peripheral devices of the system, according to some embodiments of the present invention;

FIGS. 8-12 are schematic illustrations of a propulsion unit of the system, including a spiral shaft assembly, according to some embodiments of the present invention;

FIGS. 13-19 are schematic illustrations of the propulsion unit with an alternative shaft assembly, according to some embodiments of the present invention;

FIGS. 20-22 are schematic illustrations of elements of a base of the system, including a retraction band, according to some embodiments of the present invention;

FIGS. 23-26B are schematic illustrations of a coupling mechanism of the system, according to some embodiments of the present invention;

FIGS. 27A-29B are schematic illustrations of adhesion mechanisms of the system, according to some embodiments of the present invention;

FIG. 30 is a schematic illustration of a mechanism indicating priming of a hypodermic needle of the system, according to some embodiments of the present invention;

FIGS. 31A-31C are schematic illustrations of mechanisms for sensing priming and skin contact of the system, according to some embodiments of the present invention;

FIGS. 32A-32E are schematic illustrations of drive plate configurations, according to some embodiments of the present invention; and

FIGS. 33A, 33B, 34A and 34B are schematic illustrations of mechanisms for sealing the drive and delivery sections, according to some embodiments of the present invention.

### DETAILED DESCRIPTION

It is to be understood that the invention and its application are not limited to the system and methods described below or to the arrangement of the components set forth or illustrated in the drawings, but are applicable to embodiments that may be practiced or carried out in various ways.

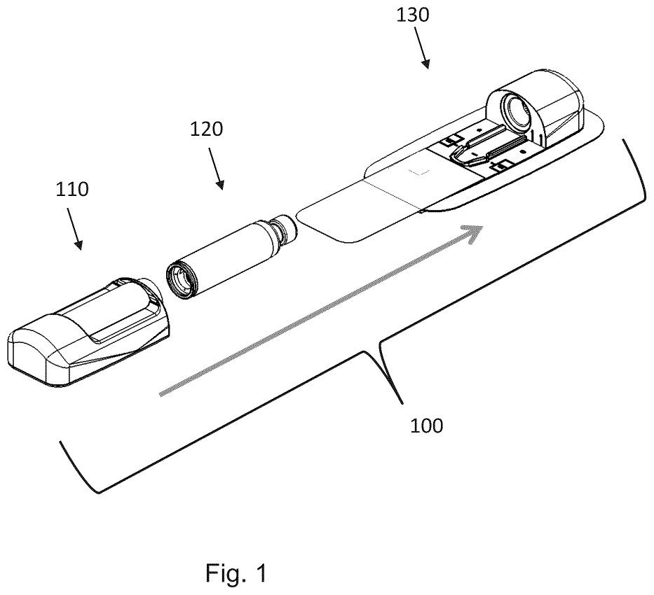

FIG. 1 is a schematic illustration of a system 100 for fluid delivery, according to some embodiments of the present invention. The system 100 includes three main sections or components: a drive section 110, a cartridge 120, and a delivery section 130. In typical usage, the cartridge 120 is fit into the drive section 110 (also referred to herein as the “reusable part”), which is then mounted onto the delivery section 130 (also referred to herein as the “disposable section”). Fluid in the fluid cartridge 120 is dispensed through the delivery section 130, after which the cartridge is refilled or replaced by separating the drive and delivery sections.

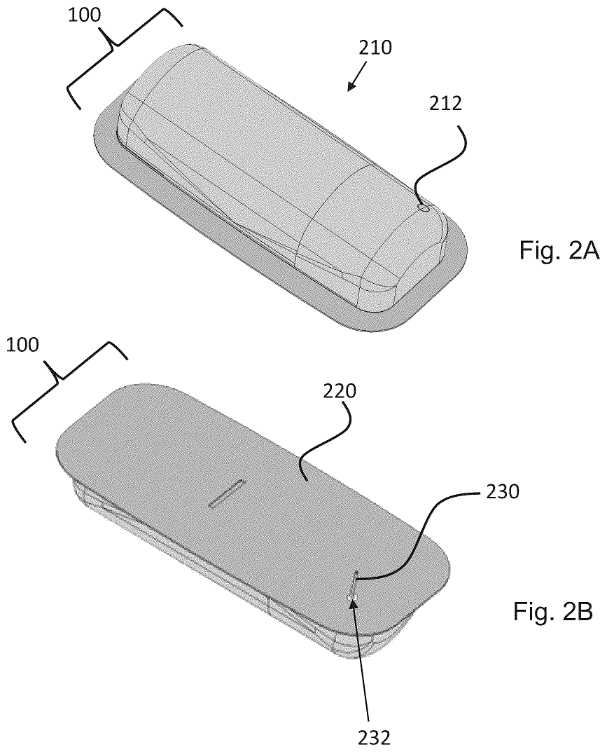

FIGS. 2A and 2B are schematic illustrations of the respective top side 210 and bottom side 220 of system 100, when the system is assembled, that is, with the drive and delivery sections joined together. A typical application of the system 100 is as a wearable device for automated drug injection, such as an insulin pump. The top side 210 may include a needle trigger port 212, through which a hypodermic needle (and/or cannula) 230 (referred to herein as a needle/cannula 230) may be added, the needle/cannula 230 being shown passing through an injection opening 232 at the bottom side 220 of the system. For an insulin pump application, the system 100 may be affixed to the skin of a patient, who may wear the system to apply multiple injections over time. For such an application, the delivery section 130 as well as the cartridge 120 are typically disposed after each use in order to ensure sterility. The bottom side 220 includes an adhesive for adhering to a patient's skin, as described further hereinbelow. Typically, both the adhesive side and the needle trigger port 212 are elements of the delivery section 130 of the system.

It is to be understood that for some alternative applications, such as applications that provide fluid delivery without injections, the delivery section 130 may be reused, that is, it is not necessarily intended to be disposed after a single use. For such alternative applications, the cartridge may alternatively be refillable. Examples of such applications include e-cigarettes and adhesive applicators. Hereinbelow, considering the non-limiting medical applications requiring disposal of the delivery section (i.e., “single-use” applications), the delivery section 130 is also referred to as the disposable section and the drive section 110 is referred to as the reusable section.

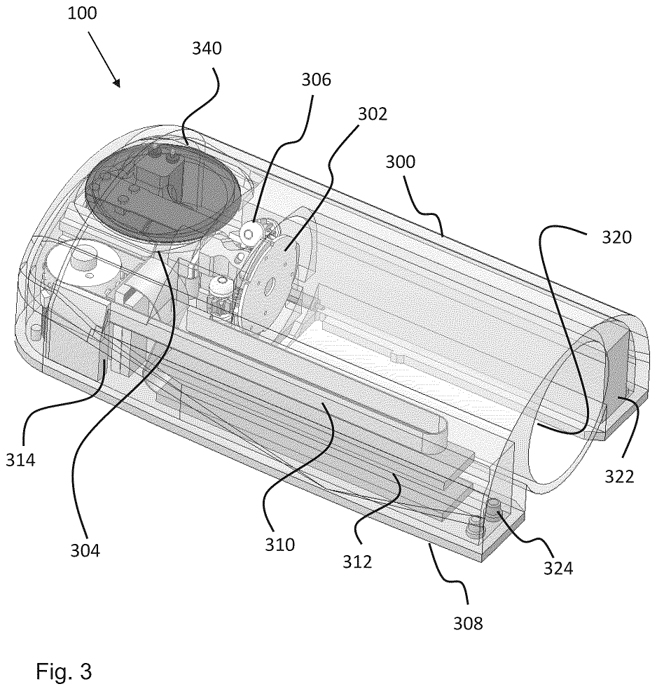

FIG. 3 is a schematic illustration of the drive section 110, which has a cover 300. The figure presents the cover 300 in a semi-transparent form so that internal components are visible. These components may include a propulsion unit 302, having a motor 304 and wheels 306. Hereinbelow, the propulsion unit is also referred to as a “propulsion device.” In addition, the drive section typically has a base 308 on which are mounted components that may include a flex cable 310, that is, a band such as flat flex cable (FFC) or a flexible printed circuit (FPC). The flex cable 310 is also referred to herein as retraction band, as one of its functions may be to retract the propulsion unit. The base 308 may also include a printed circuit board (PCB) 312, and an encoder 314. The PCB 312 typically includes a controller (not shown) or similar processor, including memory storing instructions described further hereinbelow. The PCB 312 may also include a communications device or driver for external communications, for example with an external controller, such as a mobile device, which may be configured to send instructions to the controller and to provide a user with status notifications related to operation of the system.

The cover 300 of the drive section includes a cartridge port 320 into which a plunger end of a cartridge 120 is placed (as described further hereinbelow). Additional components of the drive section may include a power source, i.e., a battery 322, battery charger contacts 324, and a buzzer and/or display 340, which may be used to convey messages to a user, such as errors or a notification that the cartridge is empty. (If the battery is meant to be disposable after a single use, it may also be located in the disposable, delivery section of the system, or it may be located in either section for applications that are not single use.)

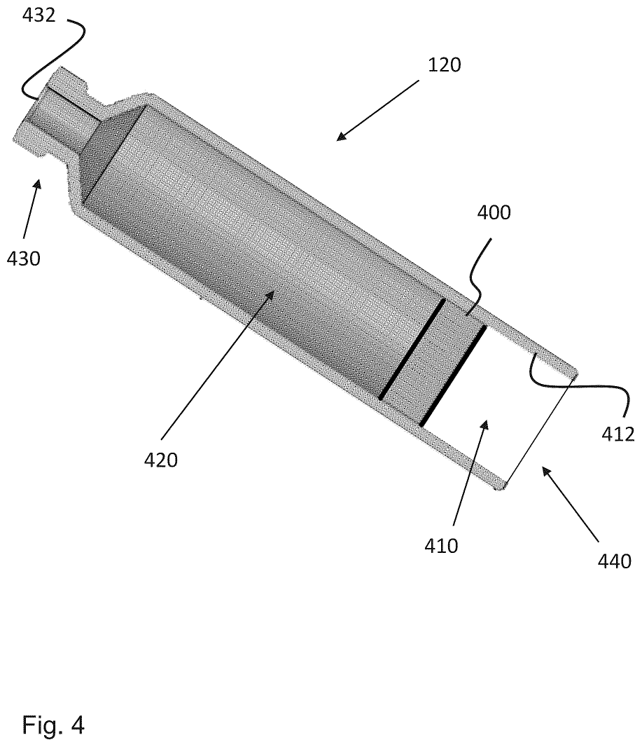

FIG. 4 is a schematic illustration of a fluid cartridge 120 of the system 100 for fluid delivery. The cartridge includes a cartridge plunger 400, which is a flexible plug located in a barrel 410 of the cartridge and fit against an inner wall 412 of the cartridge.

When the plunger 400 is pushed forward, it advances forward in the barrel 410, along the central axis of the cartridge, and fluid 420 is ejected from the cartridge through a cartridge delivery end 430. As indicated, the cartridge fluid delivery end 430 is at an end of the cartridge opposite a plunger end 440. A syringe in the delivery section of the system may be configured to pierce a seal 432 of the delivery end 430 to access the fluid.

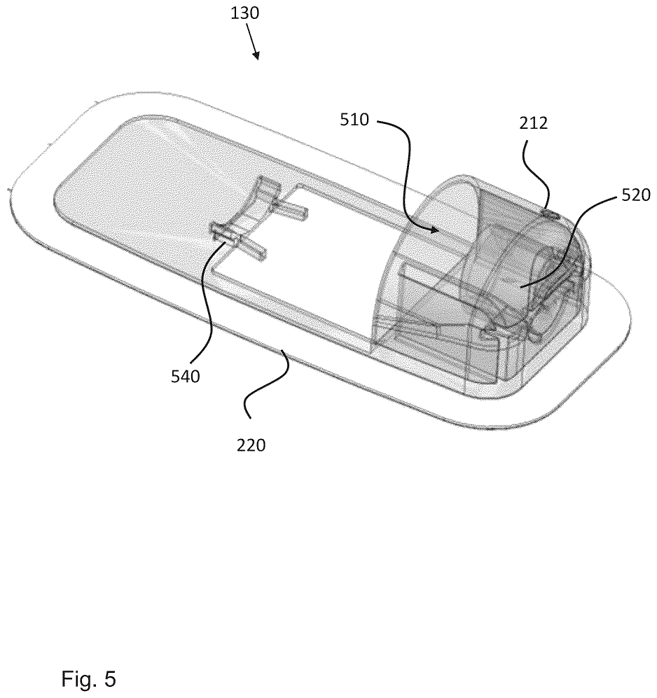

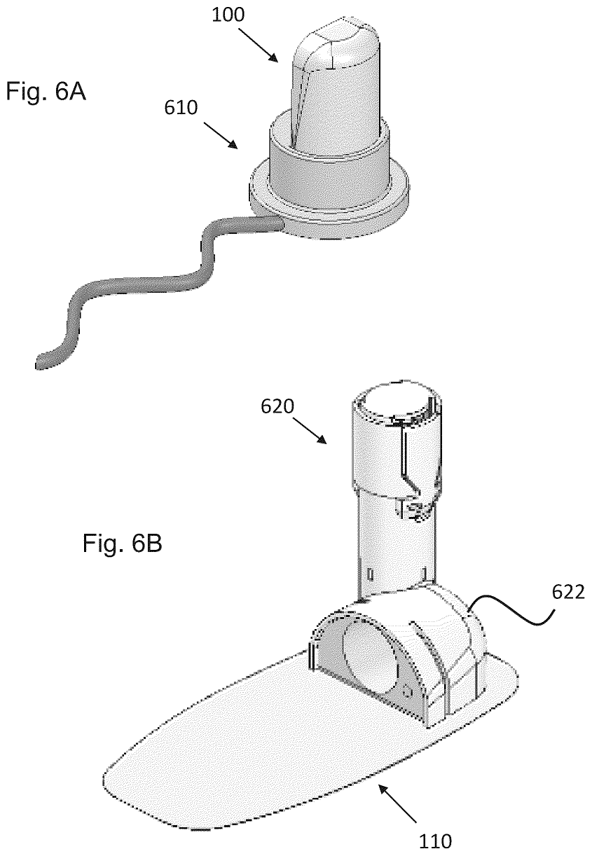

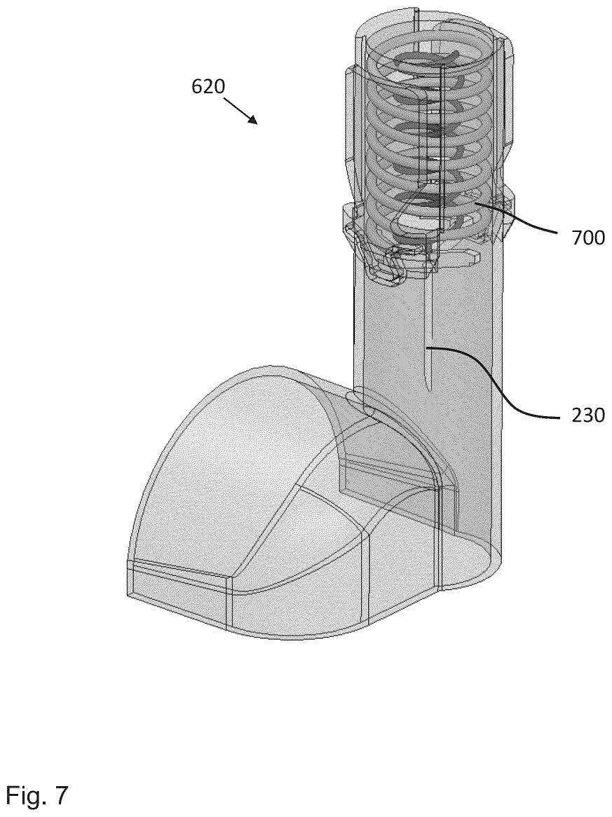

FIG. 5 is a schematic illustration of the delivery section 130 of the system 100 for fluid delivery. Two elements of the delivery section, as noted above, may be the needle trigger port 212 and the adhesive bottom side 220. Additional components that may be provided with the delivery section are a cartridge delivery end port 510, which receives the cartridge delivery end 430. Upon insertion of the cartridge into the port 510, the seal of the cartridge's delivery end is typically pierced by a syringe 520, typically connected to tubing 522 of the delivery section. The cartridge needle and connected tubing may be connected to the needle and/or cannula 230 by a needle injector described hereinbelow with respect to FIGS. 6-7. The delivery section 130 may also include a coupling connector 540, also described in more detail hereinbelow.

FIGS. 6A and 6B show peripheral devices of the system 100 for fluid delivery. FIG. 6A shows a charger 610 for recharging the battery of the system. The system 100 is inserted into the charger 610, such that the cartridge charger contacts 324 described above are in contact with contacts of the charger. Alternatively, or additionally, the system 100 may include an inductive charging coil so as to be charged by inductive charging.

As described above, the system may communicate with an external device, such as a mobile device with an appropriately configured app that communicates instructions, such as dosing quantities, to the system. The mobile device app may also provide error messages to a user, for example, notifying the user of a low battery status of the system, an event requiring recharging of the system (or battery replacement). The mobile device app may also interact with the system to send an instruction for advancing the propulsion unit to prime the needle before attachment to a user's skin. For example, before the user affixes the system to his skin, the mobile device app may request that the user approve ejection of an additional drop of fluid from the needle. Upon receiving the user approval (e.g., by the user clicking “ok” on a screen of the app), the mobile device may send an instruction to the system to propel the propulsion unit forward a slight amount (e.g., 0.5 mm) in order to eject an additional drop from the needle. The user may then be asked to confirm the ejection of a drop, i.e., successful priming of the needle. After confirming the priming, the user may then receive a notification that the system may be attached to the skin

FIG. 6B shows a needle injector 620 that includes a needle injector cover 622, which may be providing to fit over the delivery section to inject a needle for intramuscular or subcutaneous fluid delivery.

FIG. 7 is a schematic illustration of the needle injector 620, showing a spring mechanism 700 by which the needle/cannula 230 may be injected, as described above.

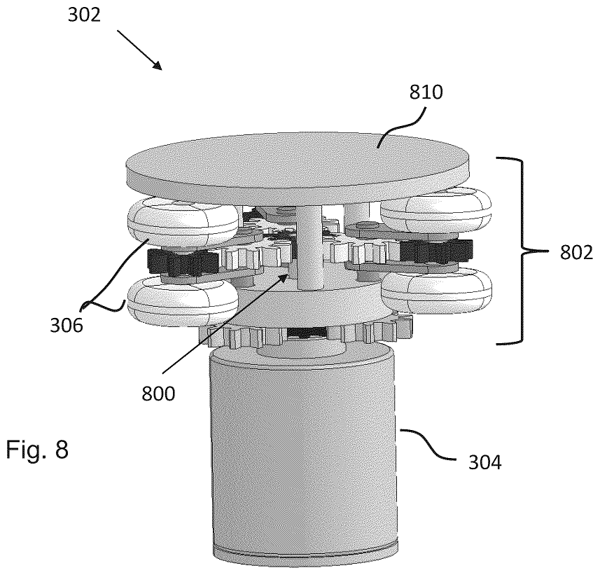

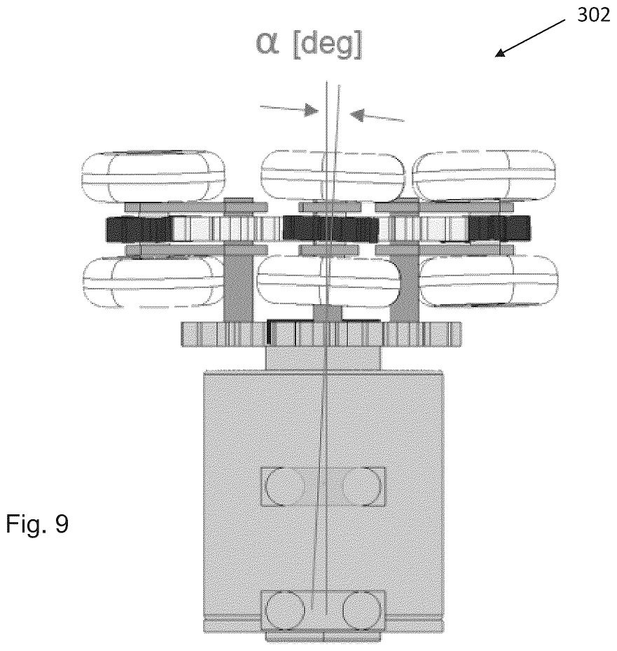

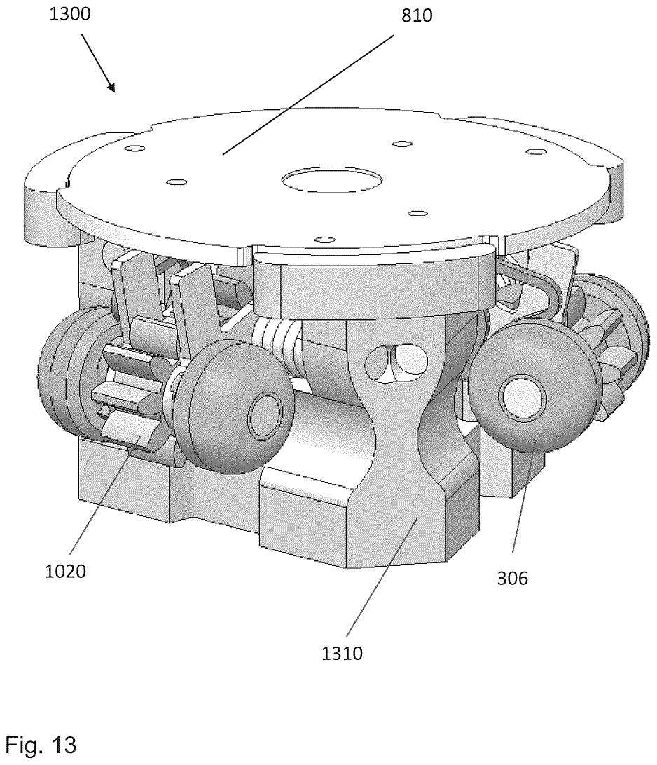

FIG. 8 is a schematic illustration of the propulsion unit 302 of the system 100 for fluid delivery. The bidirectional motor 304 of the propulsion unit drives the wheels 306 of the propulsion unit, which propel the propulsion unit forward in the barrel 440 of the cartridge 120. The motor 304 has a shaft 800, about which is mounted a shaft assembly 802. The motor is configured to fit inside the cartridge, with the motor shaft aligned coaxially with the cartridge (as described above with respect to FIGS. 3-4). The wheels 306 are mounted on a shaft assembly 802, which includes several gears described further hereinbelow and which is mounted around a motor. The shaft assembly 802 shown in FIG. 8 is configured for spiral propulsion of the propulsion unit and is therefore also referred to herein as the spiral configuration of the shaft assembly, or more simply, the “spiral” shaft assembly 802. The spiral shaft assembly is represented in FIGS. 8-12. In the spiral configuration, the wheels 306 are mounted on axes that are tilted by an angle α that is greater than 0 but less than 90 degrees with respect to the motor shaft, as shown in FIG. 9. An alternative configuration of the shaft assembly, referred to herein as a direct shaft assembly 1300, is described hereinbelow with respect to FIGS. 13-19.

Both the spiral and direct configurations of the shaft assembly include a drive plate 810. When the propulsion unit is first positioned in the barrel of the cartridge, that is, when the drive and delivery sections are joined, the drive plate 810 contacts the cartridge plunger, and may be configured to push the cartridge plunger forward slightly. The initial push of the plunger overcomes the static friction that was previously built up between the plunger and the cartridge wall (i.e., following manufacture of the pre-filled cartridge). The subsequent static friction is lower, thereby reducing the force that the propulsion unit must apply when driving the plunger by self-propulsion. The initial push applied on the plunger by joining the drive section and the delivery section may be configured to eject a small amount of fluid from the cartridge, priming the ejection needle (described further hereinbelow). In FIGS. 9-11, the drive plate of the shaft assembly has been removed to show additional components of the shaft assembly.

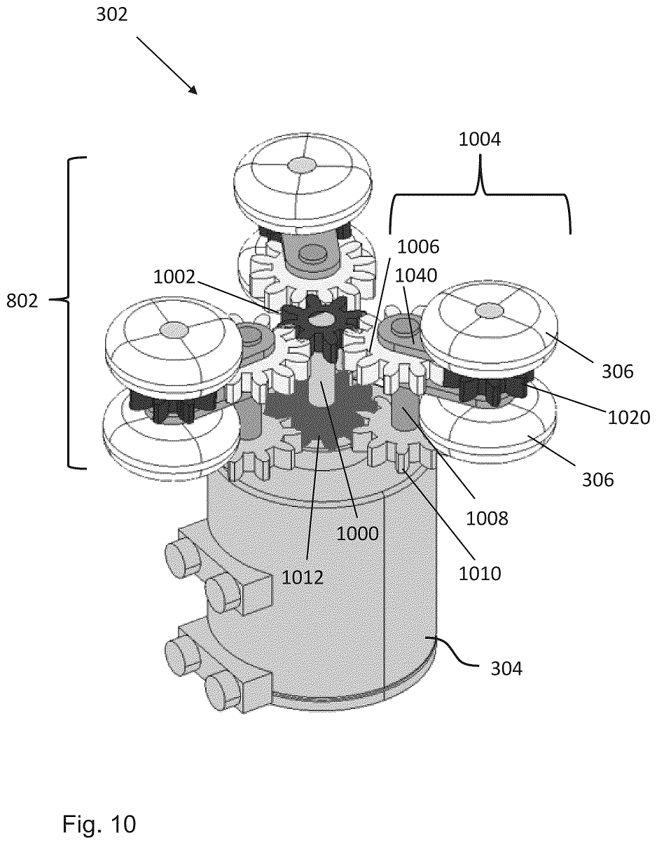

FIG. 10 is a perspective view of the propulsion unit 302 with the spiral shaft assembly 802. As described above, the propulsion unit includes the motor 304, which has a motor shaft 800. The shaft assembly 802 (like the alternative shaft assembly 1300, described below) is centered around the motor shaft 800. A motor gear 1002 of the shaft assembly 802 is affixed to the motor shaft 800 and turns together with the motor shaft. The motor gear 1002 is in turn meshed with secondary gears 1006 of respective wheel extensions 1004. As shown, the shaft assembly includes a plurality of wheel extensions 1004, which are positioned around the motor shaft, typically in a symmetric fashion, that is, with similar spacing between each wheel extension (for example, in a range of +/−10%).

Each secondary gear 1006 rotates about a secondary gear axle 1008. The secondary gear axle 1008 is tilted from an axis that is parallel to the motor shaft (i.e., tilted from an axis in a plane of the motor shaft, as shown in FIG. 9). Typically, a gripping gear 1010 is also mounted on the secondary gear axle 1008, or otherwise configured to rotate with the secondary gear, and is meshed with a fixed gear 1012. The fixed gear 1012 is positioned at the base of the motor shaft and mounted to the motor, such that the motor shaft passes through the fixed gear.

The traction of gripping gear 1010 on the fixed gear inhibits rotation of the gear motor 304 against the direction of rotation of the wheel extensions. That is, the motor does not rotate relative to the cartridge.

Each wheel extension 1004 includes at least one and typically two wheels 306. (More wheels may provide greater traction.) Each wheel or wheel pair has a common wheel gear 1020. As shown, each wheel gear 1020 meshes with a respective secondary gear 1006 of its common wheel extension, and each secondary gear also meshes with the motor gear 1002. The gears 1002, 1006, 1012 and 1020 (i.e., the motor gear, secondary gear, fixed gear and wheel gears) may be of different sizes, such that different turn ratios may be achieved, meaning that the wheels do not necessarily turn at the same rate as the motor shaft. Typically, the motor gear is smallest, such that the wheels turn more slowly than the motor gear. Note that all the gears of the spiral shaft assembly are shown as being spur gears, but they may have helically tilted grooves to facilitate the tilting of the wheels described above. It is to be understood that the configuration of gears shown is exemplary, and gear arrangements may include additional gears or alternative types of gears (e.g., helical) for transferring torque from the motor shaft to the wheel gears.

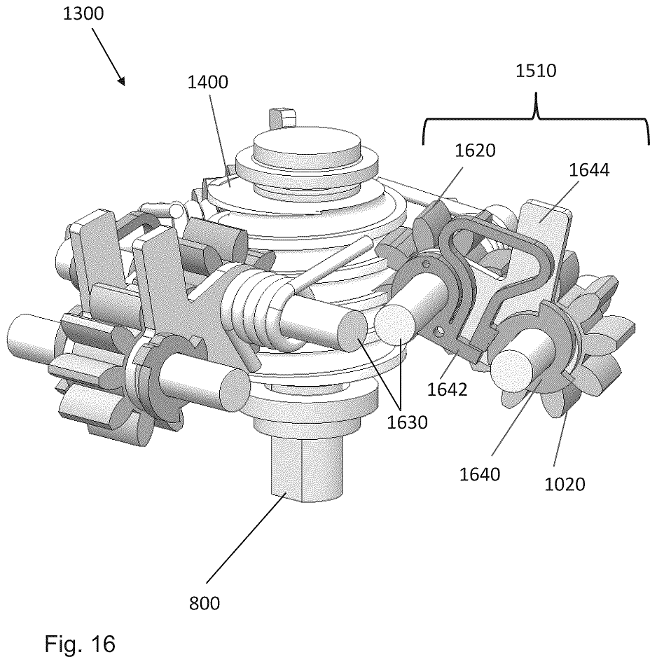

Also indicated in FIG. 10 are wheel extension plates 1040. Such plates may include elements that may be included in the spiral wheel extension 802, in the same manner that they are included in the direct wheel extension 1300, as shown in FIG. 16, described below. These elements may include a connecting plate 1630, which connects the axis of the wheels to the secondary gear axle. Additional elements of the extension plates 1040 may include a ratchet gear 1640 and a pawl spring 1642, as well as a torsion spring (1520), or similar device, these elements being described further hereinbelow.

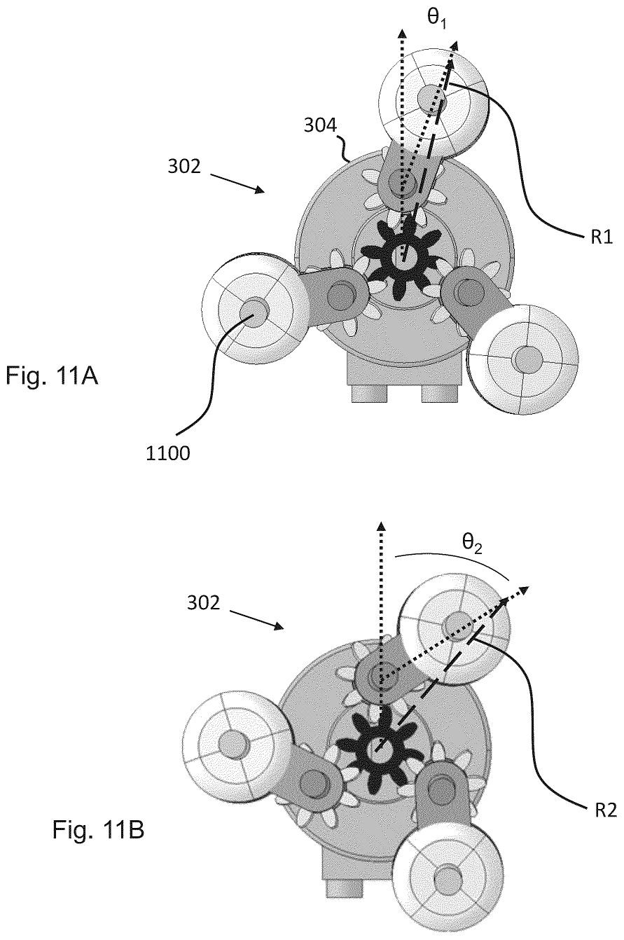

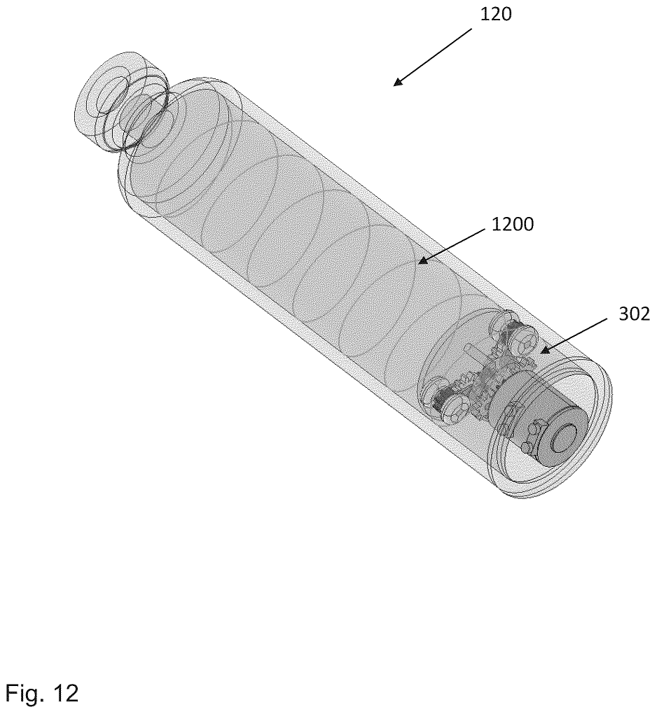

FIGS. 11A and 11B are top views of the propulsion unit 302, showing the gears of the spiral shaft assembly 802 above the motor 304. The wheel extensions are shown unfolded in FIG. 11A (to contact the cartridge wall) and folded in FIG. 11B (the position for entering and exiting the cartridge). Also shown are the wheel axles 1100 of the spiral shaft assembly. As described above, the wheel axles are tilted from the direction of axes that would be parallel to the motor shaft, so that as the wheels turn, the shaft assembly progresses into the cartridge barrel in a spiral pattern, shown below with respect to FIG. 12. As shown in FIG. 11A, when the wheels are unfolded to contact the inner wall of the cartridge, they are typically still slightly folded by an angle θ₁ with respect to a line directly extending from the motor shaft to the secondary gear. In this position, the maximum radius of the shaft assembly, including the wheel extensions, is indicated as R1. In FIG. 11B, the wheel extensions are folded, releasing their grip from the cartridge inner wall; the wheels extensions are shown folded by an angle θ₂, and the radius is shown as R2, which is less than R1.

FIG. 12 is a view of the cartridge 120 and of a spiral movement of the spiral shaft assembly 802 as the propulsion unit 302 progresses forward in the cartridge.

FIG. 13 is a view of the alternative shaft assembly described above and referred to herein as the direct shaft assembly 1300 The wheels 306 of the direct shaft assembly, as well as their wheel gears 1200, are mounted on axes that are orthogonal to the motor shaft, as opposed to the tilted positioning of the axes of the wheels of the spiral shaft assembly 802. Gears of the direct shaft assembly 1300 are held in place with a direct shaft assembly frame 1310 that also holds the drive plate 810 in place (as is also the case for the spiral shaft assembly 802).

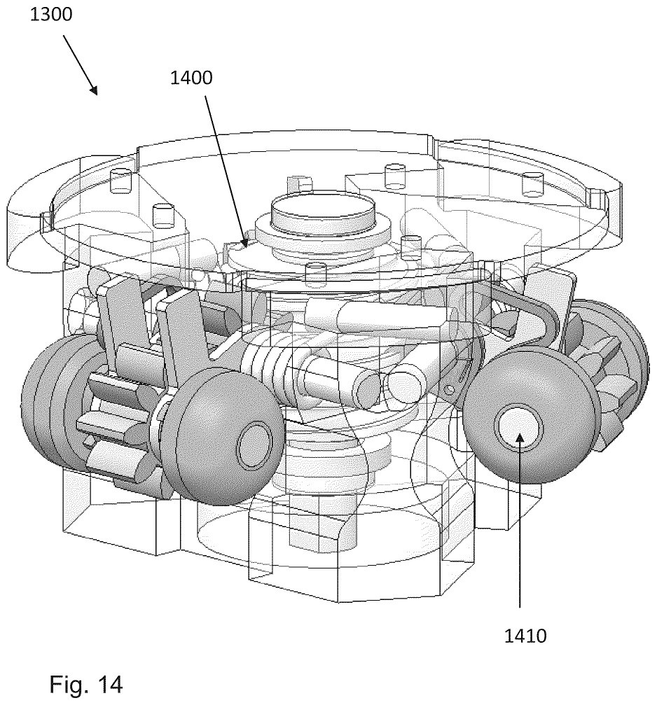

FIG. 14 is a view of the direct shaft assembly 1300 with the shaft assembly frame 1310 shown as transparent, in order to show the gear structure of the direct shaft assembly, including a direct motor shaft gear 1400. As opposed to the motor gear 1002 of the spiral shaft assembly 802, the direct motor shaft gear 1400 is configured to mesh with a secondary gear at a 90 angle. The direct motor shaft gear 1400 is typically a worm gear, as shown, or any other type of gear transferring rotation by 90 degrees, such as a bevel gear. Consequently, the wheel axles 1410 of the direct shaft assembly 1300 are orthogonal to the motor shaft. (Each wheel axle 1410 is furthermore parallel to a plane that is tangent to the point of contact of the wheel and the cartridge.)

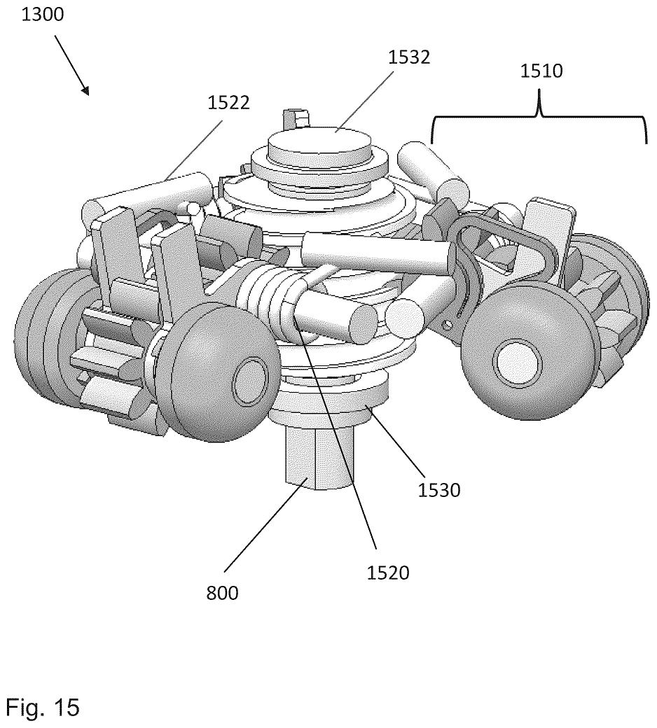

FIG. 15 is a further view of the direct shaft assembly 1300, showing additional elements. Like the spiral shaft assembly 802, the direct shaft assembly 1300 is positioned around the motor shaft 800 of the propulsion unit. Like the spiral shaft assembly 802, the direct shaft assembly 1300 also has multiple wheel extensions, indicated as wheel extensions 1510. Typically, each wheel extension has a torsion spring 1520 applying pressure to “open” the wheel extension to a configuration in which the wheels are farther from the motor shaft. The torsion springs are affixed to the shaft assembly frame 1310 by torsion spring braces 1522. Also shown are the shaft assembly bottom bearing 1530 and the shaft assembly top bearing 1532. It should be noted that like the direct shaft assembly 1300, the spiral shaft assembly 802 described above has its own torsion spring 1520 that similarly applies pressure to “open” each wheel extension to a configuration in which the wheels contact the inner wall of the cartridge. In the open (“unfolded”) position, the torsion springs maintain pressure on the wheel extensions to ensure a level of friction necessary for the wheels to grip the cartridge wall and thereby propel the propulsion unit forward as the wheels rotate.

FIG. 16 is a further view of the direct shaft assembly 1300, showing additional elements of the wheel extensions 1510. These elements include a secondary gear 1006, which meshes with both the direct motor shaft gear 1400 and with the wheel gear 1020, such that rotation of the motor shaft gear 1400 rotates a secondary gear 1620, which in turn rotates the wheel gear 1020. The secondary gear 1620 may be identical to the secondary gear 1006 of the spiral shaft assembly, described above (and similarly may include multiple secondary gears on a secondary axle 1630, for example when different gearing ratios are desired). In addition, it is to be understood that the wheel extensions 1004 of the spiral shaft assembly, like the wheel extensions 1510, typically include a ratchet gear 1640 affixed to the axis of the wheel and wheel gear, and a pawl spring 1642 affixed to the axis of the secondary gear. A connecting plate 1644 also typically connects the two axes. In typical operation, the propulsion unit, regardless of the type of shaft assembly, is inserted into a cartridge at the plunger end when the wheel extensions are “closed,” such that the wheels do not touch the inner walls of the cartridge. The motor then rotates the motor gear, which rotates the secondary gear. Due to the torsion spring, rotation of the secondary gears first lifts the wheel extensions, unfolding the wheel extensions until the wheels contact the inner wall of the cartridge. The wheels only begin to rotate after the wheels are in contact with the inner wall of the cartridge, such that the torsion springs can open the wheel extensions no further (but continue to apply pressure, as described above).

When the propulsion unit is to be retracted from the cartridge, reverse rotation of the motor gear causes a similar reverse rotation of the secondary gears. However, as the secondary gears rotate, the ratchet gears interlock with the pawl springs, preventing rotation of the wheel gears. Instead, the wheel extensions fold, separating the wheels from the cartridge wall.

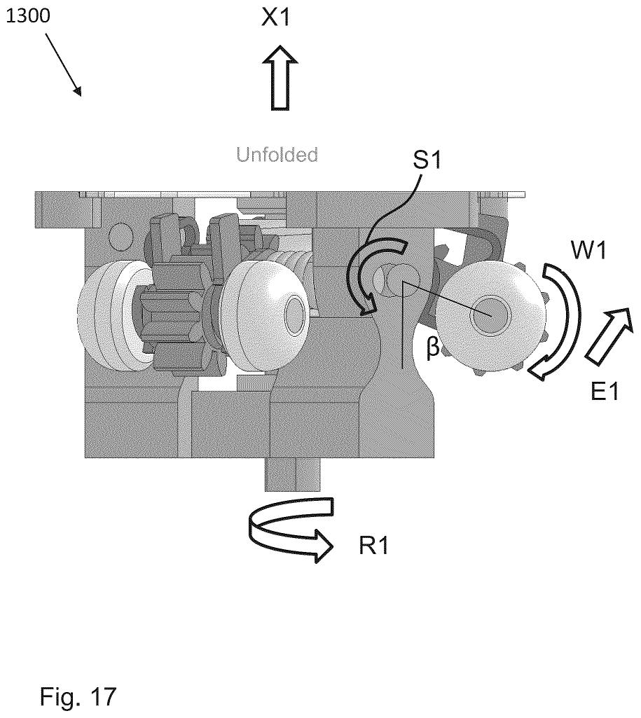

FIG. 17 shows the initial rotation R1 of the motor shaft, causing a rotation S1 of the secondary gears. Due to the torsion spring, the secondary gears first lift the wheels, indicated by arrow E1. The wheels only begins to rotate (in the direction of W1) after the wheel extensions reach an open position, at which the wheels are in contact with the inner wall of the cartridge. The rotation of the wheels (W1) advances the propulsion unit into the cartridge in direction X1.

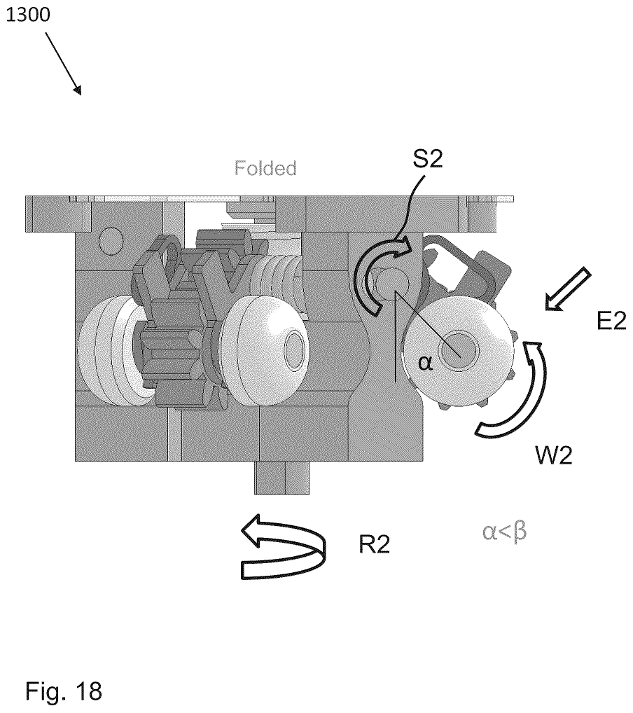

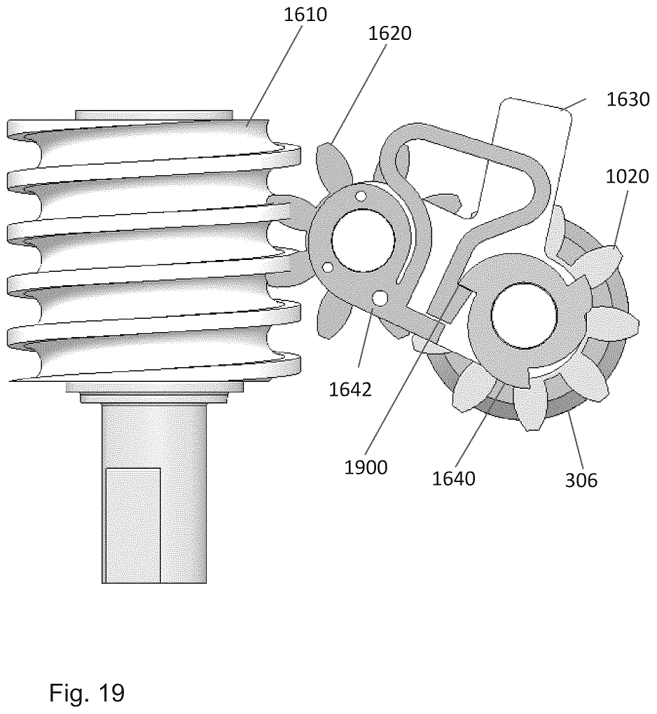

When the motor shaft rotates in the opposite direction, indicated in FIG. 18 as R2, the secondary gears rotate in the opposite direction S2. This initially causes a slight rotation of the wheels (W2) until the ratchet and pawl are locked. Further rotation of the wheels is then not possible, and further rotation of the secondary gears causes the wheel extensions to close, separating the wheels from the inner wall of the cartridge. The elements of the wheel extension are shown in clearer detail in the side view of FIG. 19, which shows the contact 1900 of the ratchet and pawl, preventing backwards rotation of the wheel. Although the FIGS. 17-19 show an example of the direct shaft assembly, the spiral shaft assembly may include the same elements, causing the same steps of operation, that is, steps of first opening the wheel extensions (due to the torsion spring), followed by rotation of the wheels, followed by reverse rotation of the motor gear causing the wheel extensions to close (due to locking of the ratchet and pawl spring).

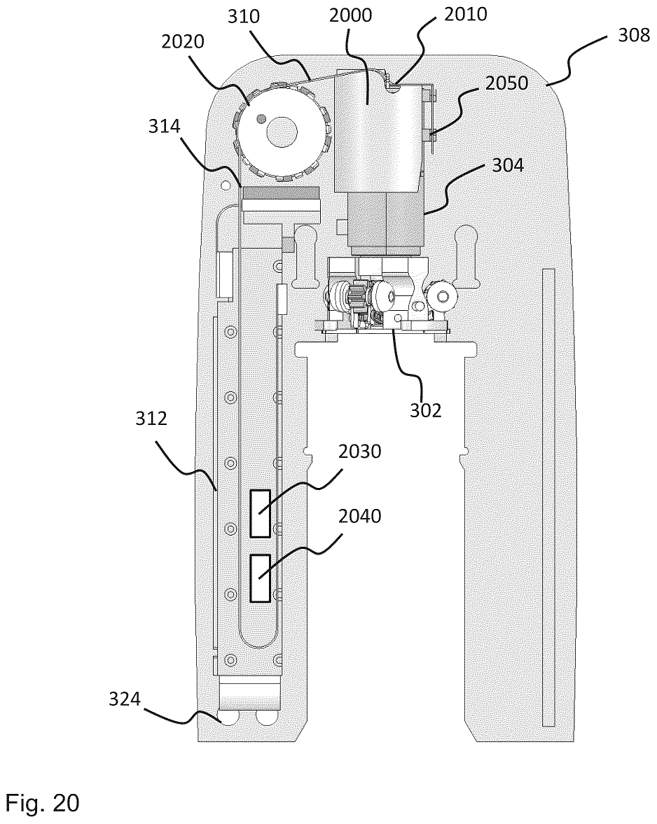

FIGS. 20-22 are schematic illustrations of a retraction mechanism of the system 100. Shown in the figures is the base 308 of the drive section, on which elements described above are positioned, including the propulsion unit 302, the propulsion unit motor 304, the flex cable 310, the PCB 312, and the battery charging contacts 324. Also shown is a propulsion unit receptacle 2000, which holds the propulsion unit before the propulsion unit advances into the cartridge. The propulsion unit receptacle 2000 is positioned such that when the cartridge is inserted into the drive section, the wheel extensions of the propulsion unit are positioned in the barrel of the cartridge behind the plunger.

The flex cable 310 is also referred to herein as a “retraction band 310,” as one of its functions is to retract the propulsion unit back into the receptacle 2000. The flex cable 310 is affixed to the propulsion unit typically at a flex cable-to-motor connection point 2010 at the back of the propulsion unit. As the propulsion unit advances into the cartridge, the flex cable is pulled into the cartridge with the propulsion unit.

In some embodiments, a retraction wheel 2020 is rotated in a first direction by the flex cable as the flex cable is pulled into the cartridge. The retraction wheel 2020 may be spring-operated or motor-operated to provide a retraction mechanism for subsequently rotating in the opposite direction, thereby pulling the flex cable and retracting the propulsion unit from the cartridge. Operation of the retraction wheel, as well as the motor of the propulsion unit, is typically controlled by a controller 2030. When the propulsion unit is to be retracted, the controller first operates the motor 304 of the propulsion unit to close the wheel extensions, separating the wheels from the inner wall of the cartridge. The retraction wheel 2020 then pulls the flex cable, which pulls the propulsion unit back into the receptacle 2000. Alternatively, the retraction wheel may have its own motor, which may receive a signal from the controller to rewind, in order to retract the propulsion unit.

As described above, the controller may also be programmed to receive instructions from an external device, such as a smart phone, by means of the communications device 2040, which may also be embedded in the controller. The external device may be configured to send such instructions, which may include, for example, instructions regarding quantity of a fluid to eject at a given time and/or instructions to retract the propulsion unit. Upon receiving input indicative of a fluid quantity to eject, the controller may trigger delivery of a signal to the motor to advance the propulsion unit by a distance calibrated to eject the indicated fluid quantity (i.e., translating a unit of volume to a unit of distance).

The retraction wheel 2020 may also include coding for the encoder 314 to measure distance moved by the propulsion unit, for example by measuring degrees of rotation of the retraction wheel. The measured distance may be measured both as the propulsion unit moves forward and when it is pulled back

The flex cable 310 may also include control wires connected to the power supply and/or the controller to supply power (e.g., two wires having a DC voltage differential, such as +5V and 0V) to the motor, thereby controlling forward or back motion of the motor. The connections of the control wires from the flex cable to the motor are indicated by flex connection 2050, which has multiple pins to permit bidirectional control of the motor.

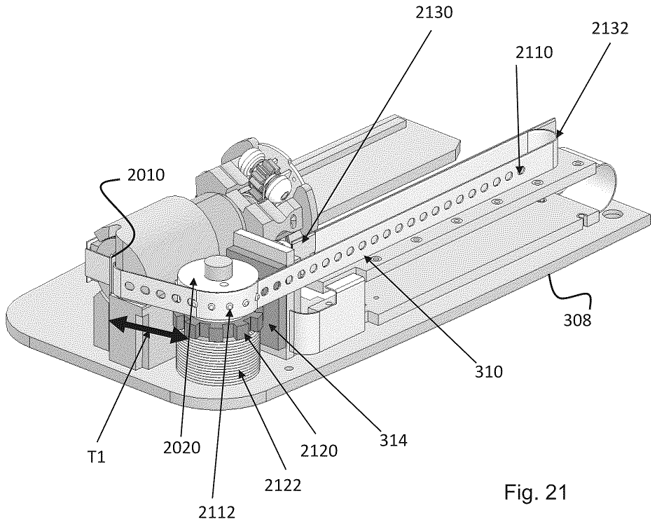

Details of the flex cable 310 and of the retraction wheel 2020 can be seen in the perspective view of FIG. 21. The flex cable 310 may have holes 2110 that fit into pins 2112 of the retraction wheel. As the flex cable advances into the cartridge, it pulls the pins of the retraction wheel thereby rotating the retraction wheel in the forward direction. Subsequently, the retraction wheel may be triggered, as described above, to rotate in the opposite direction, such that the pins pull the flex cable, which in turns pulls and retracts the propulsion unit. An arrow T1 in the figure indicates tension generated in the flex cable where it is taut between the pins of the retraction wheel 2020 and the flex cable connection point 2010.

The retraction wheel may also include an encoding gear 2120 with encoding to measure degrees of rotation that may be read by the encoder 314. The retraction wheel may also include a spring mechanism 2122 for providing the force for subsequent retraction of the propulsion unit. The flex cable 310 is typically fixed to the drive section base at a base connector 2130, which provides signals for the control wires of the flex cable, as described above. The flex cable is initially folded at a bend 2132, such that the total length of the flex cable is sufficient for the flex cable to be pulled into the cartridge to the entire extent of the cartridge barrel (as the fluid is ejected).

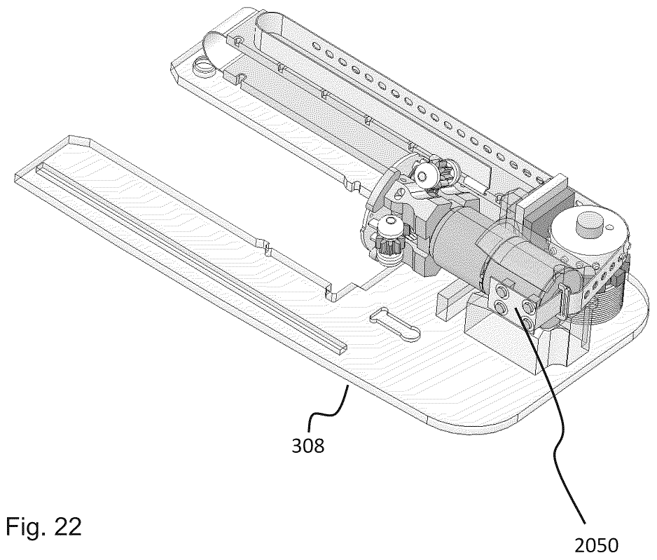

FIG. 22 is a perspective schematic illustration of the base 308 of the drive section of the system, with the base rotated from the view of FIG. 21, in order to better show the motor connection 2050. As shown, the connection may have four contacts for activating the motor (typically a stepping motor) in both directions.

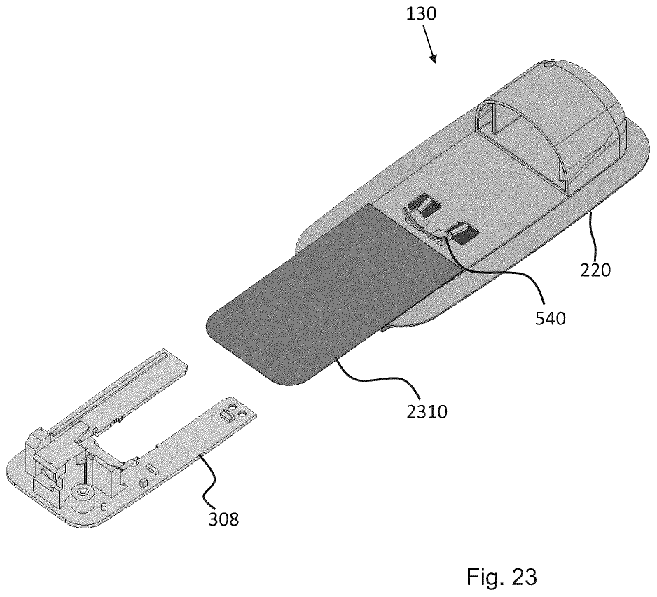

FIG. 23 is a perspective schematic illustration of the base 308 of the drive section of the system, aligned to connect with the delivery section 130 of the system. As described above, the delivery section also may have an adhesive bottom, by which the system may be attached to a patient. A peel-away adhesive cover 2310 may cover a top adhesive patch that binds the drive section and delivery section together and which is also positioned on the delivery section (the top adhesive patch being hidden from view in the figure by the adhesive cover). The delivery section also may include the coupling connector 540 (mentioned above with respect to FIG. 5), which interlocks with the drive section.

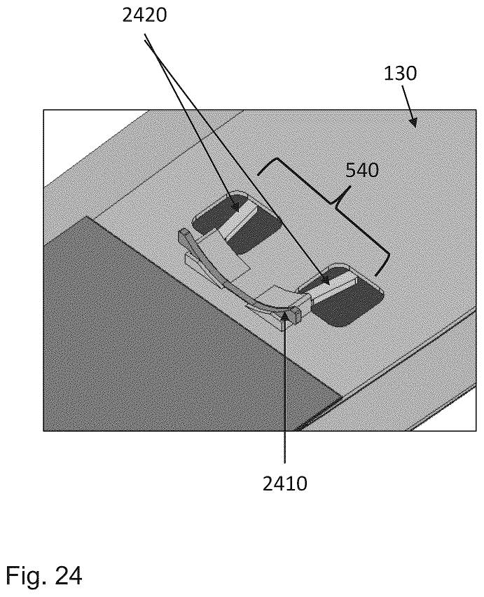

As shown in FIG. 24, the coupling connector 540 of the delivery section 130 typically includes two elements, a coupling plate 2410 and a set of one or more axial snaps 2420 (or a similar interlocking connector).

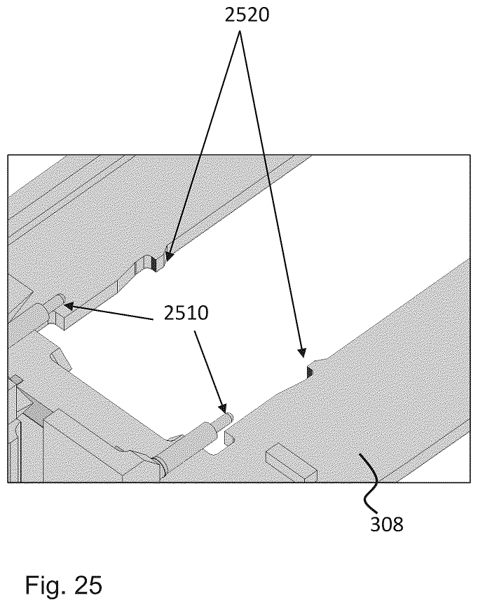

As shown in FIG. 25, the base 308 has one or more coupling sensors 2510 (e.g., spring pins) and snap stoppers 2520 configured to interconnect with the respective axial snaps 2420.

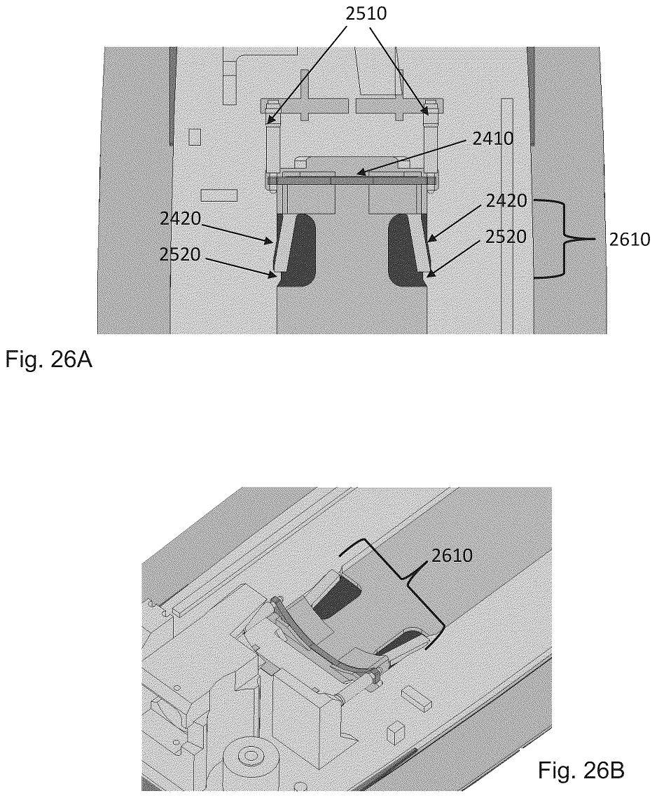

FIG. 26A is a schematic illustration of a top view of the coupling connector 540 interlocked with the complementary elements of the base 308 of the drive section. As indicated, the coupling plate 2410 is in contact with the coupling sensors 2510. The coupling sensors 2510 are typically connected to the controller, which determines whether or not there is continuity between the coupling sensors. Continuity provides an indication to the controller that the coupling plate is in position and the drive section and the delivery section are therefore interlocked.

The drive section and the delivery section may be configured to be detached from each other in order to insert a new, prefilled cartridge. A user may insert a new cartridge first into the delivery section, which pierces the seal (i.e., “septum”) of the cartridge, so that fluid can flow through tubing of the delivery section to a hypodermic needle. Next, the user attaches the drive section to the cartridge and pushes the drive and delivery sections together (so that, for example, the coupling connector and axial snaps may interconnect). The process of connecting the two sections may also press the propulsion unit against the cartridge plunger, priming the fluid tubing and hypodermic needle.

The controller is typically programmed to identify the connection, by sensing an electrical continuity indicated by the coupling sensors, and in response to trigger the motor to unfold the wheel extensions and drive the propulsion unit forward to contact the plunger.

The controller is also typically further programmed to identify a disconnection of the drive section from the delivery section, by sensing a lack of electrical continuity indicated by the one or more coupling sensors 2510, and in response to trigger the motor to fold the wheel extensions, which triggers the retraction wheel to retract the propulsion unit (typically by releasing the spring-loaded torque of the retraction wheel). As described above, the coupling sensor may include one or more electrical contacts in one part (either the delivery section or the drive section) and one or more electrically conductive elements in the other part configured to measure contact between the drive and delivery sections. Alternatively, or additionally, the coupling sensor may include a Hall sensor in one part that is aligned with a magnet in the other part, or a Near Field Communication (NFC) sensor in one part that is aligned with an Ntag in the other part. Any of these, or other known coupling sensors may be used in to detect a detachment between the drive and delivery sections and to trigger retraction of the propulsion unit.

FIG. 26A also shows a complete locking mechanism 2610 providing locking by interconnection of the snaps 2420 with the stoppers 2520, to help maintain a connection between the drive section and the delivery section. FIG. 26B shows the same interconnections from a perspective view. The interconnections are typically designed to release the two parts when the drive section is lifted from the delivery section or the second is bent below the drive section.

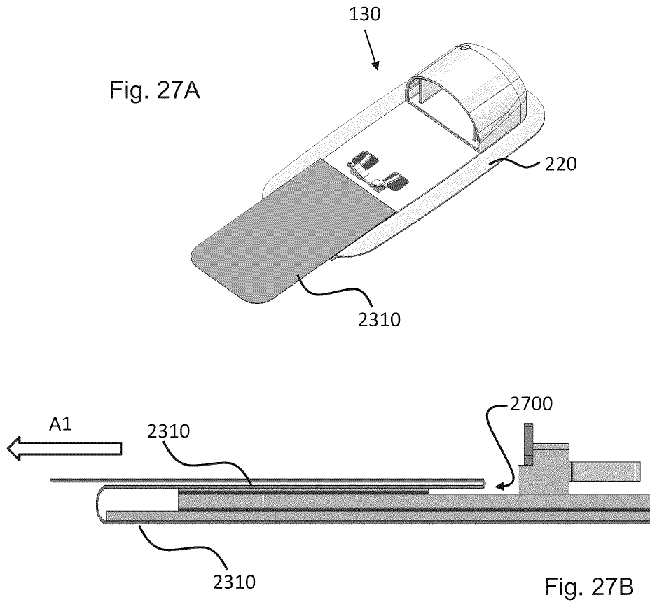

FIGS. 27A and 27B are schematic illustrations of an adhesion mechanism of the system 100. As described above, the delivery section 130 typically has an adhesive bottom side 220, for attaching the system to a user's body after the drive and delivery sections are connected. In addition, the delivery section may have a top-side adhesive patch 2700 that ensures a secure connection between the drive and delivery sections.

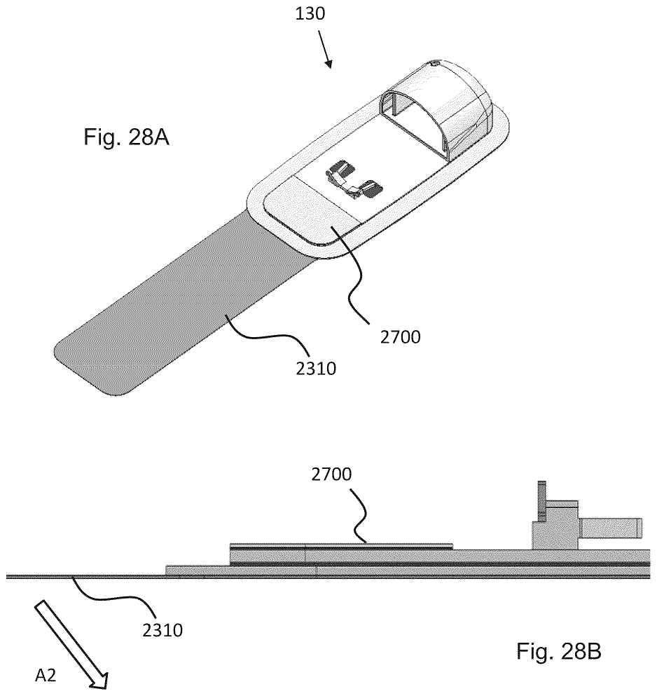

As shown in the figures, a peel-off cover 2310 initially covers the top-side adhesive patch 2700. FIG. 27B is a side view of the delivery section, showing that the peel-off cover may be folded on the top side, where it covers the top adhesive patch 2700, and also extends to cover the adhesive patch underside of the delivery section. When the peel-off cover is in place over the top adhesive patch, the drive section may be affixed to the delivery section by the coupling connector described above. (The peel-off cover should not be removed before connection of the drive section, because then the top adhesive patch would be exposed, preventing a user from sliding the two parts together.) After the parts are connected, a user may pull on the peel-off cover in the direction of the arrow A1, thereby exposing the top adhesive patch 2700, causing the two parts to stick together. An exposed view of the top adhesive patch is shown in FIGS. 28A and 28B. When the top adhesive patch 2700 is exposed, the two parts are typically already joined, such that the top adhesive patch is hidden from view.

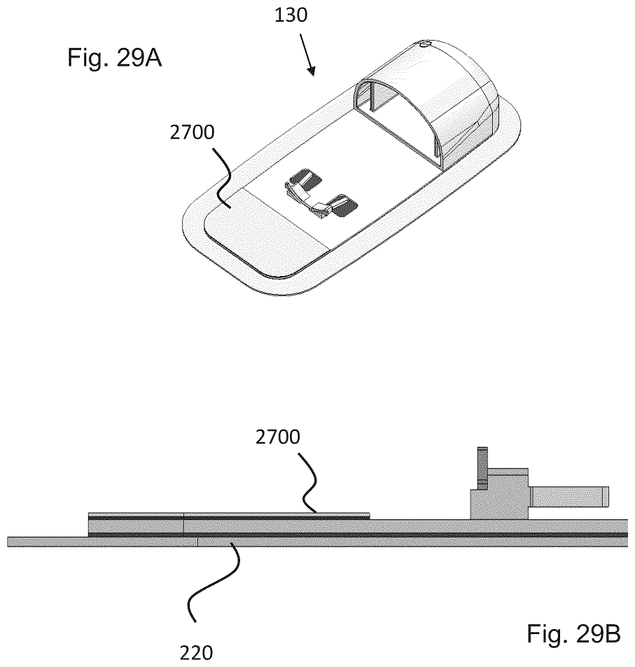

After the drive and delivery sections are attached to each other, with the peel-off cover extracted from between the two parts as shown in FIGS. 28A and 28B, the user can pull off the peel-off cover from the underside adhesive patch by pulling the peel-off cover in the direction of the arrow indicated as A2. The adhesive bottom side 220 is then exposed, as shown in FIGS. 29A and 29B, such that the system can be affixed to the body of a patient. Typically, when the system is subsequently pulled from the skin of the patient, the delivery section is bent below the drive section, causing the adhesive patch binding the two parts to release the parts from each other.

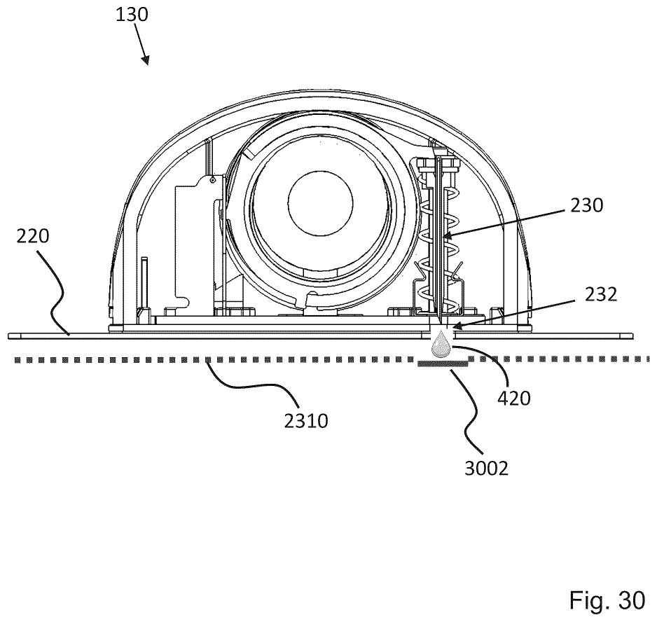

FIG. 30 is a schematic illustration of a mechanism for indicating priming of the needle/cannula 230 of the system 100. The underside 220 of the delivery section 130 includes an opening 232 for injection of the needle/cannula 230. Before injection, the system is configured to advance the propulsion unit against the plunger in order to “prime” the system, ejecting a small amount of fluid 420, e.g., a drop of fluid, to ensure that no air remains in the tubing or needle/cannula before injection. To provide a user indication of priming, the underside 220 of the delivery section may include a litmus patch 3002, such as a small patch of litmus paper. The litmus patch may also be provided affixed to the peel-away adhesive cover 2310. The litmus patch absorbs the drop of fluid ejected by priming and “reacts,” for example by changing colors, or otherwise showing fluid absorption, thereby indicating proper priming. The controller may be configured to receive user input confirming the priming in order to proceed with operation (e.g., injection and fluid ejection).

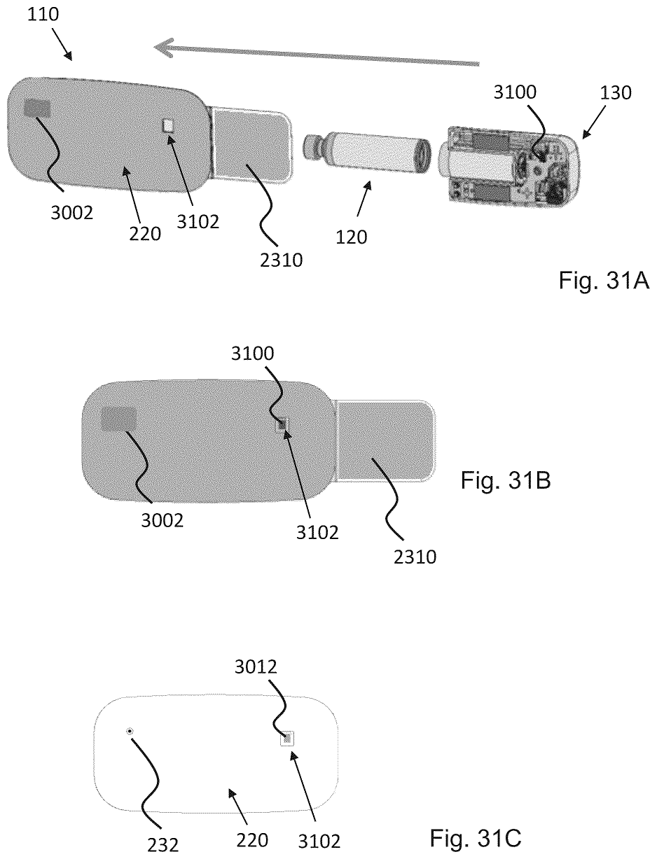

FIGS. 31A-31C are schematic illustrations of a mechanism for sensing skin contact of the system. As indicated in FIG. 31A, the drive section may include a proximity sensor 3100. The sensor is configured to measure proximity and/or contact between the system and a patient's skin. When the drive and delivery sections are joined, as indicated in FIG. 31B, the proximity sensor is positioned above a window 3102 in the underside of the delivery section. FIG. 31C shows the system with the adhesive peel-away cover 2310 removed. The sensor is typically connected to the controller, described above, providing a signal to the controller indicative of the skin contact (i.e., the “proximity”). The controller may be configured to wait for such a signal, verifying system attachment, before proceeding with subsequent steps (e.g., injection of needle/cannula, ejection of fluid, etc.) Subsequent detachment of the system from the skin provides a detachment signal. The controller may be configured to react to such a trigger signal of detachment by retracting the propulsion unit to its initial position in the propulsion unit receptacle (for example, by triggering the retraction of the flex cable, described above). This retraction, as described above, ensures that the propulsion unit is not in the delivery section when the drive section is separated.

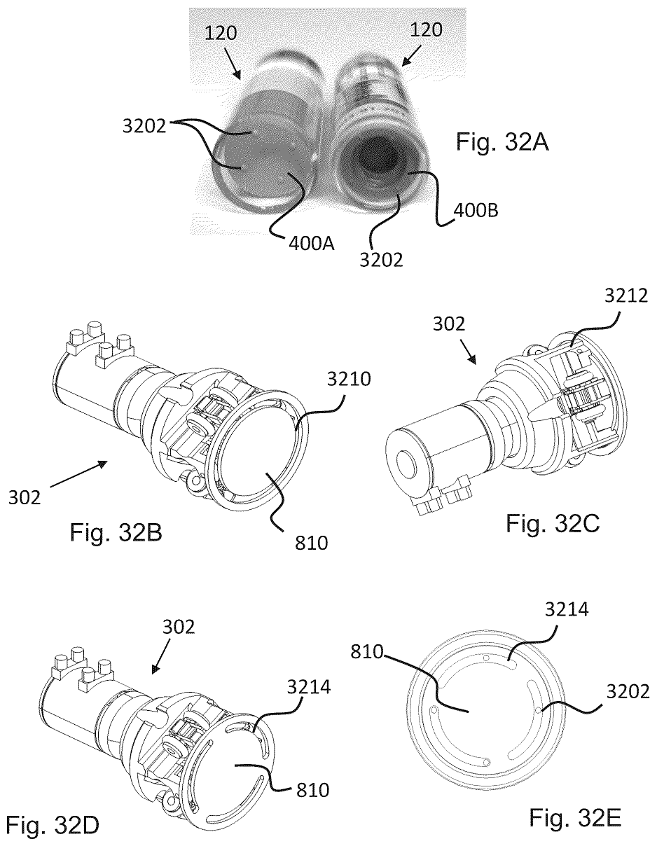

FIGS. 32A-32E are schematic illustrations of mechanisms of drive plate configurations of the propulsion unit. As indicated in FIG. 32A, there may be multiple configurations of the plunger of the cartridge 120, such as a plunger 400A and a plunger 400B. In most configurations, the plunger face has protrusions 3202 that may absorb some of the pressure of the propulsion unit plate 810, reducing the consistency of operation. To overcome this problem of plunger protrusions, grooved cutouts in the plate may be provide, indicated in FIG. 32B as a full cutout 3210, and in FIGS. 32D and 32E as partial cutouts 3214. The edge of the plate with the full cutout 3210 may be supported by an edge support 3212, as shown in FIG. 32C.

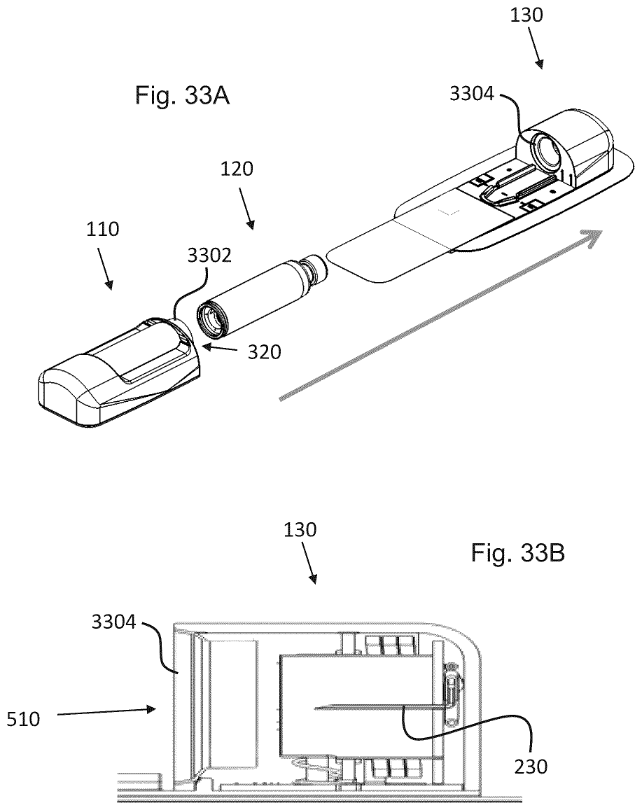

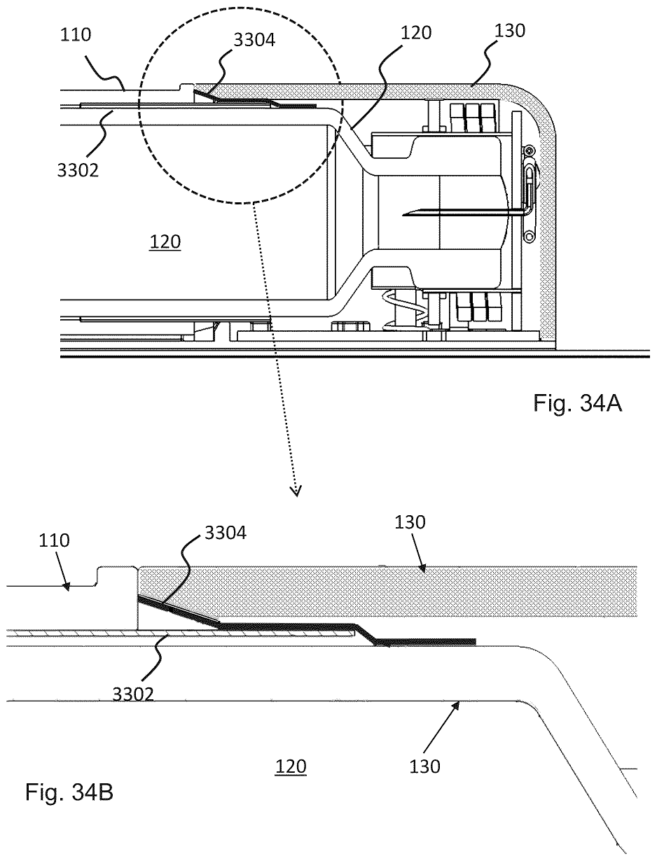

FIGS. 33A, 33B, 34A and 34B are schematic illustrations of mechanisms for creating a seal between the drive section 110 and the delivery section 130, when the two sections are joined to enclose the cartridge 120. As shown in FIGS. 33A-33B, the drive section cartridge port 320 may include a stiff, circular sleeve 3302 (such as ceramic or metal), and the delivery section cartridge end port 510 may include a flexible, circular seal 3304. The sleeve and the seal encircle the cartridge when the cartridge is inserted in each section. As indicated in FIGS. 34A-34B, interconnection of the drive and delivery sections, when the two sections are locked together, partially inserts the sleeve between the cartridge and the seal, preventing fluid and gases from entering the drive section.

### Examples

Example one, that is, a first exemplary embodiment of the invention described herein, is a system including:
1) a propulsion unit (302) for driving a plunger (400) in a barrel (410) of a cartridge (120) to eject a fluid (420) from a delivery end (430) of the cartridge;
2) a drive section (110) comprising a drive section cartridge port (320), configured to receive a plunger end (440) of the cartridge and comprising a propulsion unit receptacle (2000) configured to hold the propulsion unit and from which the propulsion unit enters the cartridge barrel; and
3) a delivery section (130) comprising a cartridge delivery end port (510) configured to receive the delivery end of the cartridge and to release the fluid from the cartridge when the propulsion unit drives the plunger forward.

An example 2 includes the features of example one and the propulsion unit further includes:
1) a motor (304) having a motor shaft (800) aligned coaxially with the cartridge;
2) a motor shaft gear (1002, 1400) affixed to the motor shaft; and
3) multiple wheel extensions (304, 1510) arranged around the motor shaft, each wheel extension comprising a secondary gear (1006, 1620), one or more wheels (306) and a wheel gear (1020) mounted to an axle (1100, 1410) of the one or more wheels. The rotation of the motor shaft in a first direction unfolds the wheel extensions until the wheels of the multiple wheel extensions are in contact with an inner wall (412) of the cartridge. When the wheels are in contact with the inner wall of the cartridge, rotation of the motor shaft in the first direction rotates the wheels in contact with the inner wall to propel the propulsion unit forward.

In an example 3, example 2 includes an additional feature of the secondary gear of each wheel extension being configured to mesh with the motor shaft gear and with a respective wheel gear of the wheel of the wheel extension.

In an example 4, either of the examples 2 or 3 includes an additional feature of the multiple wheel extensions being positioned in an approximately symmetrical arrangement around the motor shaft.

In an example 5, any of the examples 2-4 includes an additional feature of the motor being controllable to rotate in a reverse direction opposite to the first direction, wherein each wheel extension further comprises a ratchet gear (1640) and a pawl spring (1642) that lock during a backward rotation of the wheels and wherein, after locking of the ratchet gear and the pawl spring, rotation of the motor shaft in the reverse direction folds the wheel extensions.

In an example 6, any of the examples 2-5 includes an additional feature of a spring (1520) applying torque to unfold the wheel extension away from the motor shaft when the motor rotates in the first direction. In an example 7, the spring is a torsion spring connected to the wheel extension to apply the torque to unfold the wheel extension.

In an example 8, any of the examples 2-7 includes an additional feature of the secondary gear (1620) rotating on an axle (1630) orthogonal to the main shaft, such that rotation of the wheels when in contact with the inner wall of the cartridge advances each wheel in a direction parallel to the motor shaft. In an example 9, the motor shaft gear is a worm gear (1400).

In an example 10, any of the above examples 2-7 includes an additional feature of the propulsion unit having a fixed gear (1012) mounted to the motor and through which the motor shaft extends. Each wheel extension also has a gripping gear (1010), configured to rotate with the respective secondary gear of the wheel extension, and meshed with the fixed gear (1012). The wheel gear of the wheel extension is mounted on an axle (1100) tilted by less than 90 degrees with respect to the motor shaft, and rotation of the wheels when in contact with the inner wall of the cartridge advances the wheel extensions in the cartridge barrel in a spiral motion.

In an example 11, any of the above examples includes an additional feature of a controller (2030) configured to receive an input indicative of a fluid quantity and, responsively, to trigger delivery of a signal to the motor to advance the propulsion unit by a distance calibrated to eject the indicated fluid quantity.

In an example 12, any of the above examples includes an additional feature of a flex cable (310) connecting the propulsion unit to a retraction mechanism (2020) in the drive section. The retraction mechanism is configured to be triggered to pull the flex cable, pulling the propulsion unit into the drive section for reuse.

In an example 13, the features of example 12 further include the retraction mechanism including a spring-loaded wheel (2020).

In an example 14, the features of either of examples 12-13 further include an encoder (314), wherein the retraction mechanism has coding that is readable by the encoder, and wherein the encoder measures, based on displacement of the flex cable, a distance moved by the propulsion unit and a corresponding quantity of fluid ejected.

In an example 15, the features of any of examples 12-14 further include a coupling sensor (324) configured to measure contact between the drive and delivery sections and to trigger retraction of the propulsion unit when the drive and delivery sections are separated.

In an example 16, the features of example 15 further include the coupling sensor being configured to trigger folding of wheel extensions of the propulsion unit before triggering retraction of the propulsion unit.

In an example 17, the features of either of examples 15-16 further include a controller configured to receive a first signal from the coupling sensor indicative of detachment of the drive and delivery systems and responsively to issue a second signal triggering retraction of the propulsion unit.

In an example 18, the features of any of examples 15-17 further include the coupling sensor having one or more electrical contacts in the drive section and one or more complementary electrically conductive elements in the delivery section, or a Hall sensor in the drive section and a magnet in the delivery section, or an NFC sensor in the drive section and an Ntag on the delivery section, in order to detect detachment of the drive section from delivery section and to trigger the retraction of the propulsion unit.

In an example 19, the features of any of examples 12-18 further include the flex cable being further configured to provide power to the motor from a power source (322).

In an example 20, the features of any of examples 1-19 further include a locking mechanism (2610), configured to lock the drive and delivery sections together, and to release the lock when the delivery section is bent below the drive section.

In an example 21, the features of examples 20 further include the locking mechanism having axial stoppers positioned in the drive section that interlock with axial snaps in the delivery section.

In an example 22, the features of any of examples 1-21 further include an adhesive patch (2700) for affixing the drive section to the delivery section. The adhesive patch is covered by a folded adhesive cover (2310), preventing the drive and delivery sections from being adhered together before the drive section is aligned on top of the delivery section, and the folded adhesive cover is removable by pulling to expose the adhesive patch when the drive section is aligned with the delivery section.

In an example 23, the features of example 22 further include the adhesive patch being a first adhesive patch and a second adhesive patch (220) being provided for affixing the delivery section to a patient's skin. The folded adhesive cover also covers the second adhesive patch is removable from the second adhesive patch before or after being removed from the first adhesive patch.

In an example 24, the features of any of examples 1-23 further include the cartridge being made of glass, wherein the propulsion unit has wheels for advancing the propulsion unit into the cartridge barrel, and wherein the wheels are made of a material having a coefficient of fiction (COF) with glass of >0.05.

In an example 25, the features of example 24 further include wheel material that is any one of: machinable glass ceramic; tetragonal zirconia polycrystals, ZrO2-Y2O3; zirconia (ZrO2); aluminum oxide-alumina (Al2O3); silicon carbide (SiC); stainless steel; nickel (Ni); glass reinforced plastic; or glass.

In an example 26, the features of any of examples 1-25 further include the fluid being a therapeutic drug and the delivery section having tubing (520) connecting the fluid delivery end to a needle/cannula (230) for injection into a patient.

In an example 27, the features of any of examples 1-26 further include an underside of the delivery section having an opening (232) for a needle/cannula (230) and a litmus patch (3002) covering the opening and configured to react (e.g., visibly) to release of the fluid during priming of the system.

In an example 28, the features of any of examples 1-27 further include a proximity sensor (3100) configured to measure proximity and/or contact between the system and a patient's skin and to trigger an alarm and/or retraction of the propulsion unit to an initial position in the propulsion unit receptacle when the system is separated from the patient's skin.

In an example 29, the features of example 28 further include a controller configured to receive a first signal from the proximity sensor indicative of attachment of the system to a patient's skin and responsively to issue a second signal indicating valid attachment.

In an example 30, the features of any of examples 1-29 further include the propulsion unit having a drive plate (810) that contacts the plunger when the propulsion unit drives the plunger and having one or more cutouts (3210, 3214) positioned to avoid plunger protrusions.

In an example 31, the features of any of examples 1-30 further include the drive section cartridge port having a stiff sleeve encircling the cartridge, and the cartridge delivery end port includes a flexible seal encircling the cartridge. Interconnection of the drive and delivery sections inserts the sleeve and the cartridge into the seal, preventing fluid and gasses from entering the drive section.

It is to be understood that the scope of the present invention includes variations and modifications thereof which would occur to persons skilled in the art upon reading the foregoing description and which are not disclosed in the prior art. Although the invention has been described in detail, nevertheless, changes and modifications, which do not depart from the teachings of the present invention, will be evident to those skilled in the art. Such changes and modifications are deemed to come within the purview of the present invention and the appended claims.

It will be readily apparent that the various methods and algorithms described with respect to the controller may be implemented by appropriately programmed general purpose computers and other types of computing devices. Furthermore, programs that implement such methods and algorithms may be stored and transmitted using a variety of media in a number of manners. In some embodiments, hard-wired circuitry or custom hardware may be used in place of, or in combination with, software instructions for implementation of the processes of various embodiments. Thus, embodiments are not limited to any specific combination of hardware and software. Transmission media include coaxial cables, copper wire and fiber optics, including wires that comprise a bus coupled to the processor. Transmission media may include or convey acoustic waves, light waves and electromagnetic emissions, such as those generated during radio frequency (RF) and infrared (IR) data communications. Instructions may be delivered from memory to the processor carried over a wireless transmission medium, and/or may be formatted according to numerous formats, standards or protocols, such as Bluetooth, TDMA, CDMA, 3G.

The following is a table of elements referenced in the above specification of the present invention:

| | |
|---|---|
| 100 | system for fluid delivery |
| 110 | drive section of system |
| 120 | fluid cartridge |
| 130 | delivery section of system |
| 210 | top side of delivery section |
| 212 | needle trigger port |
| 220 | bottom (adhesive) of delivery section (skin-facing, underside) |
| 230 | hypodermic needle and/or soft cannula |
| 232 | needle injection opening on underside of delivery section |
| 302 | propulsion unit (propulsion “unit”) |
| 304 | motor of propulsion unit |
| 306 | wheels of propulsion unit |
| 308 | base of drive section |
| 310 | flex cable (“retraction band”) |
| 312 | printed circuit board (PCB), with controller, comm port |
| 314 | encoder |
| 320 | drive section cartridge port |
| 322 | battery (“power source”) |
| 324 | battery charger contacts |
| 340 | buzzer and/or display |
| 400 | cartridge plunger |
| 410 | cartridge barrel |
| 412 | inner wall of cartridge |
| 420 | cartridge fluid (liquid, gel, etc.) |
| 430 | cartridge delivery end |
| 432 | cartridge delivery end seal |
| 440 | plunger end of cartridge |
| 510 | cartridge delivery end port of the disposable unit |
| 520 | delivery syringe |
| 522 | delivery tubing |
| 540 | coupling connector |
| 620 | needle injector |
| 622 | needle injector cover |
| 802 | shaft assembly (of spiral propulsion device) |
| 810 | shaft assembly top plate (“cover”) |
| 800 | motor shaft |
| 1002 | motor shaft gear (of spiral mechanism) |
| 1004 | wheel extensions (“extension arms”) of spiral mechanism |
| 1006 | secondary gear of spiral mechanism |
| 1008 | secondary gear axle, spiral mechanism |
| 1010 | gripping gear, spiral mechanism |
| 1012 | motor fixed gear |
| 1020 | wheel gear |
| 1040 | wheel extension plates and ratchet |
| 1100 | wheel axle of spiral shaft assembly |
| 1200 | spiral movement of shaft assembly |
| 1300 | shaft assembly of direct drive mechanism |
| 1310 | direct shaft assembly frame |
| 1400 | motor shaft gear (of direct mechanism, e.g., worm gear) |
| 1410 | wheel axles of direct mechanism |
| 1510 | wheel extensions (“extension arms”) of direct mechanism |
| 1520 | spring (torsion spring) |
| 1522 | spring brace |
| 1530 | shaft assembly bottom bearing |
| 1532 | shaft assembly top bearing |
| 1620 | secondary gear of direct mechanism |
| 1630 | secondary gear axle |
| 1640 | ratchet gear |
| 1642 | pawl spring |
| 1644 | gear connecting plate |
| 1900 | ratchet/pawl contact |
| 2000 | receptacle of propulsion unit |
| 2010 | connection of flex cable to motor of propulsion unit |
| 2020 | retraction wheel (and/or encoder wheel) |
| 2030 | computer controller (“controller”) |
| 2040 | communications device |
| 2050 | flex cable control wire connection to propulsion unit motor |
| 2110 | flex cable holes |
| 2112 | retraction wheel pins |
| 2120 | encoding gear |
| 2122 | spring of retraction wheel |
| 2130 | flex cable connection to base |
| 2132 | flex cable bend |
| 2310 | peel-away adhesive cover |
| 2410 | coupling sensor plate |
| 2420 | snaps (“axial” or similar) |
| 2510 | coupling sensor (e.g., coupling pins, or other known technologies, |
| | such as a Hall sensor aligned with a magnet, or a |
| | Near Field Communication (NFC) sensor aligned with an Ntag) |
| 2520 | snap stoppers |
| 2700 | top adhesive patch (joining drive and delivery sections) |
| 3002 | litmus patch for priming confirmation |
| 3100 | proximity and/or contact sensor |
| 3102 | proximity sensor window in bottom side of delivery section |
| 3202 | plunger bumps (protrusions) |
| 3210 | drive plate cutout |
| 3212 | drive plate edge support |
| 3214 | drive plate partial cutout |
| 3302 | sleeve of drive section cartridge port |
| 3304 | seal of delivery end port |

1. A system for fluid delivery comprising:
a propulsion unit configured to drive a plunger in a barrel of a cartridge to eject a fluid from a delivery end of the cartridge;
a drive section comprising a drive section cartridge port, configured to receive a plunger end of the cartridge and comprising a propulsion unit receptacle configured to hold the propulsion unit and from which the propulsion unit enters the barrel of the cartridge; and
a delivery section comprising a cartridge delivery end port configured to receive the delivery end of the cartridge and to release the fluid from the cartridge when the propulsion unit drives the plunger forward.

2. The system of claim 1, wherein the propulsion unit further comprises:
a motor having a motor shaft aligned coaxially with the cartridge;
a motor shaft gear affixed to the motor shaft; and
multiple wheel extensions arranged around the motor shaft, each wheel extension comprising a secondary gear, one or more wheels and a wheel gear mounted to an axle of the one or more wheels,
wherein rotation of the motor shaft in a first direction unfolds the wheel extensions until the wheels of the multiple wheel extensions are in contact with an inner wall of the cartridge,
wherein, when the wheels are in contact with the inner wall of the cartridge, rotation of the motor shaft in the first direction rotates the wheels in contact with the inner wall to propel the propulsion unit forward.

3. The system of claim 2, wherein the secondary gear of each wheel extension meshes with the motor shaft gear and with a respective wheel gear of the wheel extension.

4. The system of claim 2, wherein the multiple wheel extensions are positioned in an symmetrical arrangement around the motor shaft.

5. The system of claim 2, wherein the motor is controllable to rotate in a reverse direction opposite to the first direction, wherein each wheel extension further comprises a ratchet gear and a pawl spring that lock during a backward rotation of the wheels and wherein, after locking of the ratchet gear and the pawl spring, rotation of the motor shaft in the reverse direction folds the wheel extensions.

6. The system of claim 2, further comprising a spring applying torque to unfold the wheel extension away from the motor shaft when the motor rotates in the first direction.

7. (canceled)

8. The system of claim 2, wherein the secondary gear rotates on an axle orthogonal to a main shaft, such that rotation of the wheels when in contact with the inner wall of the cartridge advances each wheel in a direction parallel to the motor shaft.

9. The system of claim 8, wherein the motor shaft gear is a worm gear.

10. The system of claim 2, wherein the propulsion unit further comprises a fixed gear, mounted to the motor and through which the motor shaft extends, and wherein each wheel extension has a gripping gear, configured to rotate with the respective secondary gear of the wheel extension, and meshed with the fixed gear, wherein the wheel gear of the wheel extension is mounted on an axle tilted by less than 90 degrees with respect to the motor shaft, and wherein rotation of the wheels when in contact with the inner wall of the cartridge advances the wheel extensions in the barrel of the cartridge in a spiral motion.

11. The system of claim 2, further comprising a controller configured to receive an input indicative of a fluid quantity and, responsively, to trigger delivery of a signal to the motor to advance the propulsion unit by a distance calibrated to eject the indicated fluid quantity.

12. The system of claim 1, further comprising a flex cable connecting the propulsion unit to a retraction mechanism in the drive section, and wherein the retraction mechanism is configured to be triggered to pull the flex cable, pulling the propulsion unit into the drive section for reuse.

13. The system of claim 12, wherein the retraction mechanism comprises a spring-loaded wheel.

14. The system of claim 12, further comprising an encoder, wherein the retraction mechanism has coding that is readable by the encoder, and wherein the encoder measures displacement of the propulsion unit and a corresponding quantity of fluid ejected according to displacement of the flex cable.

15. The system of claim 12, further comprising a coupling sensor configured to measure contact between the drive section and the delivery section.

16-18. (canceled)

19. The system of claim 12, wherein the flex cable is further configured to provide power to a motor from a power source.

20. The system of claim 1, further comprising a locking mechanism, configured to lock the drive section and the delivery section together, and to release a lock of the locking mechanism when the delivery section is bent below the drive section.

21. The system of claim 20, wherein the locking mechanism comprises axial stoppers positioned in the drive section that interlock with axial snaps in the delivery section.

22-23. (canceled)

24. The system of claim 1, wherein the cartridge is made of glass, wherein the propulsion unit has wheels for advancing the propulsion unit into the barrel of the cartridge, and wherein the wheels are made of a material having a coefficient of fiction (COF) with glass of >0.05.

25. The system of claim 24, wherein the material of the wheels is selected from the group consisting of:
1. machinable glass ceramic;
2. tetragonal zirconia polycrystals, ZrO₂—Y₂O₃;
3. zirconia (ZrO₂);
4. aluminum oxide-alumina (Al₂O₃);
5. silicon carbide (SiC);
6. stainless steel;
7. nickel (Ni);
8. glass reinforced plastic; and
9. glass.

26. (canceled)

27. The system of claim 1, wherein an underside of the delivery section comprises an opening for a needle/cannula and further comprises a litmus patch covering the opening and configured to react to release of the fluid during priming of the system.

28. The system of claim 1, further comprising a proximity sensor configured to measure proximity and/or contact between the system and a patient's skin and to trigger an alarm and/or retraction of the propulsion unit to an initial position in the propulsion unit receptacle when the system is separated from the patient's skin.

29. The system of claim 28, wherein the system further comprises a controller configured to receive a first signal from the proximity sensor indicative of attachment of the system to a patient's skin and responsively to issue a second signal indicating valid attachment.

30. The system of claim 1, wherein the propulsion unit further comprises a drive plate that contacts the plunger when the propulsion unit drives the plunger and having one or more cutouts positioned to avoid plunger protrusions.

31. The system of claim 1, wherein the drive section cartridge port comprises a stiff sleeve encircling the cartridge, wherein the cartridge delivery end port comprises a flexible seal encircling the cartridge, and wherein interconnection of the drive and delivery sections inserts the sleeve and the cartridge into the seal, preventing fluid and gasses from entering the drive section.
